(19) United States
(12) Patent Application Publication
(10) Pub. No.: US 2024/0352207 A1
(43) Pub. Date: Oct. 24, 2024

(54) SYSTEMS AND METHODS FOR ISOLATION AND SUBSEQUENT DISPENSATION OF NATURAL PRODUCTS

(71) Applicants: Joseph Matthew McLellan, Rocklin, CA (US); Brian E. Mayers, Quincy, MA (US); Myer Berlow, Quincy, MA (US)

(72) Inventors: Joseph Matthew McLellan, Rocklin, CA (US); Brian E. Mayers, Quincy, MA (US); Myer Berlow, Quincy, MA (US)

(21) Appl. No.: 18/761,116

(22) Filed: Jul. 1, 2024

Related U.S. Application Data
(63) Continuation of application No. 16/769,315, filed on Jun. 3, 2020, now abandoned.
(60) Provisional application No. 62/585,429, filed on Nov. 13, 2017.

(51) Int. Cl. C08J 5/18; B29C 71/02; B29K 27/00; B29L 7/00

(52) U.S. Cl. CPC C08J 5/18; B29C 71/02; B29C 2071/022; B29K 2027/16; B29K 2995/0012; B29L 2007/002; C08J 2327/16

(57) ABSTRACT

Disclosed herein are systems and methods whereby raw materials are extracted, refined to a point where pure natural-product compounds are isolated and then packaged into formulation cartridges, which are then used in an extract formulator to produce reproducible natural-product extracts based on predetermined formulations

### CROSS-REFERENCE(S) TO RELATED APPLICATION(S)

This application is a continuation of U.S. patent application Ser. No. 16/763,915, filed May 13, 2020, which is a National Stage Entry of International Application No. PCT/US2018/060787, filed Nov. 13, 2017, which claims the benefit of U.S. Provisional Application No. 62/585,429, filed Nov. 13, 2017, the disclosure of which is hereby incorporated by reference in its entirety.

### BACKGROUND

Many people use natural products as supplements, cosmetics, medicine, and for recreational purposes. These products can range from fresh, dried, or cured plant materials to essential oils and extracts that are either used directly or in combination with other materials. The effects of these materials are often attributed to what is known as the entourage effect, where the effects on the user do not come from a single component of the natural product but rather than from the combination of all of the major and minor components interacting synergistically with the body at the same time. For example, the cannabis plant is known to express many different cannabinoids including Tetrahydrocannbinolic acid (THCA), Tetrahydrocannabinol (THC will be used herein for both Δ⁹-THC the major isomer and Δ⁸-THC minor isomer), Cannabidiolic Acid (CBDA) Cannabidiol (CBD), cannabinol (CBN), Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM), as well as many other related compounds (Morales, P., Hurst, D. P., & Reggio, P. H. (2017). “Molecular Targets of the Phytocannabinoids: A Complex Picture.” Progress in the chemistry of organic natural products, 103, 103-131.). Most of these cannabinoids are present in the fresh plant material in the acid forms (e.g. THCA), which convert to the “active” decarboxylated forms (e.g. THC) after drying, aging, curing, or a thermal decarboxylation process. In addition, cannabis produces other compounds including terpenes that help define the aroma and flavor of the plants, and may have a wide range of activity in the body ranging from directly interacting with receptors to regulating uptake of other compounds. Examples of such compounds include β-myrcene, a-pinene, limonene, β-caryophyllene, linalool, terpinolene, camphene, α-terpineol, phellandrene, delta-3-carene, α-humulene, pulegone, sabinene, and geraniol. Due to the entourage effect and the low concentrations of some of these components, even minor changes to composition and/or concentration can have a significant impact on the effects one may experience when they consume these natural products.

The plant materials used to make natural products are inherently variable in composition, which means that the consumer may have a variable response batch-to-batch or even dose-to-dose. Variations in composition can arise from natural variations within a plant, e.g. the top and bottom of the plant may produce different levels of chemicals based on differences in the amount of light, water, or even slight differences in the temperature. Some plant-to-plant variations are expected because it is difficult to ensure that every single portion of every single plant experiences the exact same conditions throughout its life, and these minor differences will have an impact in the specific composition. Variations can also occur from misidentification/labeling, contamination, or degradation over time. For example, there are well known strains in the cannabis industry but there are not means by which those strains are regulated or validated. So, two growers may be growing cultivars that they each call “White Widow”, but which genetically, phenotypically, and/or chemically are distinct from one another and that would produce very different responses for a consumer, especially when the variability of drying curing and extraction are factored in. What is needed is a means of producing consistent natural product extract formulations that yield reproducibly uniform chemical profiles batch-to-batch and dose-to-dose over time. Such a system is expected to be appreciated by consumers, entities trying to build national and international brands, and especially the research community where reproducibility is critical to clinical study design.

### SUMMARY

In one aspect, a natural-products extract formulator is provided. In one embodiment, the system includes:
at least two cartridges; wherein each cartridge is configured to contain a purified natural product, a mixture of purified natural products, or additives, and at least one of the natural product extracts is a pure extract that is free of other additives, carriers, solvents, viscosity modifiers, or diluents;
at least two precision dispensers, each configured to dispense the purified natural product from one of the cartridges;
a database of formulations that is located either locally or externally, and which determines how much of each purified natural product is to be dispensed to create each formulation in the database; and
at least one mixer configured to mix the purified natural products dispensed from the at least two precision dispensers; and
a controller that is configured to control dispensation of purified natural products from the natural-products extract formulator by controlling the function of the at least two cartridges, the at least two precision dispensers, the mixer, and at least one temperature within the system.

In another aspect of the current invention, a system for preparing a natural-products formulation is provided. In one embodiment, the system includes:
at least two purified natural product extracts;
a natural-products extract formulator as disclosed herein; and
a receiving container or vial;
wherein the system is configured to dispense the at least two purified natural product extracts in precise quantities and mixed at an appropriate temperature and with sufficient agitation to yield a homogenous natural product formulation.

In another aspect, a cartridge is provided that is configured to be used in a natural-product extract formulator as disclosed herein.

In another aspect, a method of manufacturing a natural-products formulation is provided and includes forming a natural-products formulation using a natural-products extract formulator and/or system as disclosed herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates an exemplary process for cannabis extracts in accordance with embodiments disclosed herein.

FIG. 2 illustrates a natural-products extract formulator in accordance with embodiments disclosed herein.

FIG. 3 illustrates an external control system in accordance with embodiments disclosed herein.

FIG. 4 illustrates an exemplary cartridge in accordance with embodiments disclosed herein, configured for use with a natural-products extract formulator.

FIG. 5 illustrates another exemplary cartridge in accordance with embodiments disclosed herein, configured for use with a natural-products extract formulator.

### DETAILED DESCRIPTION

Disclosed herein are systems and methods whereby raw materials are extracted, refined to a point where pure compounds (also referred to herein as “extracts”, “isolates”, “distillates”, and “purified natural products”)—including, but not limited to, the compounds disclosed above in the Background section—are isolated and then packaged into formulation cartridges (also referred to herein as “cartridges” or “formulation cartridges”) which are then used in an extract formulator to “print” reproducible “natural product formulations” based on predetermined formulations.

In the system and method, purified natural product extracts are provided in the cartridges, which are then used in an extract formulator to “print” a natural products formulation. “Pure” or “purified” in the present disclosure indicates a natural product compound with 90-100% (wt.) of a target compound or class of compounds. In some embodiments a single species or compound (e.g. THC or CBD) is preferred, as it allows for the precise tuning of the concentration of that component in the final product. In other cases, the full spectrum of the class of cannabinoid compounds derived from a particular cultivar are preferred, in this case the pure natural product extract would substantially only contain compounds of the cannabinoid class (i.e. substantially free of terpenes, fats, lipids, solvents, viscosity modifies, or other additives), but the cannabinoids would be present as a mixture of species. In other cases, a subset of the cannabinoids of the full spectrum may be combined to represent the “profile” of the major cannabinoids from a particular cultivar and used as a “master batch”. This may be done to ensure consistency and accuracy of the concentrations of each component in formulations prepared with this master batch. Master batching may be done to ensure accuracy and consistency of the concentrations of components in the final formulations, to capture the full spectrum of a class of compounds from a cultivar, to reduce processing costs, or for convenience. Stability of the compounds may also impact the purity, for example the THCA is the dominant cannabinoid in many cultivars, but through drying, aging, or exposure to heat THCA can convert to THC and can further convert to CBN with exposure to light and oxygen (Wang M, Wang Y H, Avula B, et al. Decarboxylation Study of Acidic Cannabinoids: A Novel Approach Using Ultra-High-Performance Supercritical Fluid Chromatography/Photodiode Array-Mass Spectrometry. Cannabis Cannabinoid Res. 2016; 1(1): 262-271. Published 2016 Dec. 1. doi: 10.1089/can.2016.0020.) In all of these cases pure will refer to either a single compound or a single class of compounds.

In some embodiments the pure compounds are comprised of >90% (wt.) of the target compound or class of compounds, in some embodiments the pure compounds are comprised of >95% (wt.) of the target compound or class of compounds, in some embodiments the pure compounds are comprised of >99% (wt.) of the target compound or class of compounds, and in other embodiments the pure compounds are comprised >99.5% (wt.) of the target compound or class of compounds. Depending on the intended application, it may be preferred to package “pure” extracts in cartridges that contain essentially one class of compounds but with well-defined ratios of components, e.g. a series of pure cannabinoid extracts that contain >90% cannabinoids but with different ratios, as examples THC to THCA, THC to CBD, and THC to CBN. Alternatively, it may be desirable to package fully characterized mixtures of cannabinoid extracts and other known components into cartridges as “master batches.”

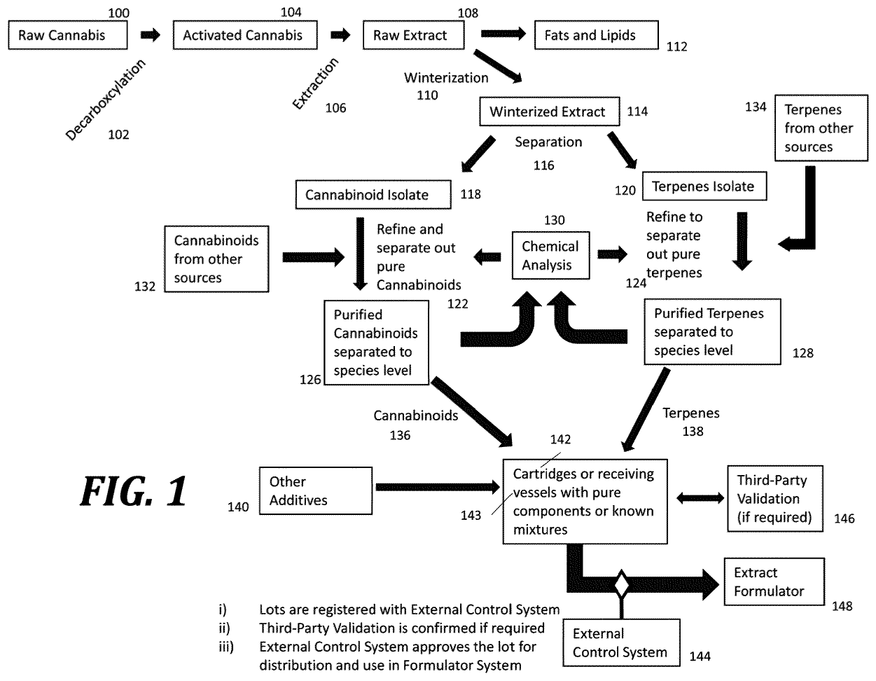

An example of the process for going from raw materials to cartridges or pre-filled receiving vials is shown below in FIG. 1. In FIG. 1, raw cannabis (100) is first subjected to a decarboxylation process (102), wherein the carboxylate forms of the cannabinoids in the raw cannabis are converted to activated cannabinoids (104); for example, conversion of THCA to THC or conversion of CBDA to CBD. In this context the “raw” cannabis is assumed to have been dried and cured during which some of the cannabinoids may have been decarboxylated, but the majority of cannabinoids will still be in the acid form. Through the application of heat the carboxylic acid is driven off as carbon dioxide leaving the active cannabinoid behind. Excessive heating may result in loss of terpenes form the raw cannabis, or further conversion of THC to CBN so the process must be optimized. An example process for decarboxylation would be heating the raw cannabis to 145 degrees Celsius for 1 hr., to yield an activated cannabis.

Extraction (106) of the activated cannabis can occur by a variety of means, but the most common means include solvent extraction with alcohol or other organic solvents, pressurized hydrocarbons, or supercritical carbon dioxide. The goal of extraction is to separate the cannabinoids and terpenes in the activated cannabis from the rest of the plant material. In a typical solvent extraction process the activated cannabis is washed with solvent, which solvates the cannabinoids and terpenes from activated cannabis, the plant materials is then separated from the solution to yield a raw extract (108). The raw extract may contain some fats and lipids (112) that may be separated from the rest of the raw extract by winterization (110). Winterization can be accomplished by a variety of means. One example process involves chilling the raw extract solution to a point where the fats and lipids condense out of solution, and then filtering the condensed fats and lipids from the raw extract solution to yield a winterized extract (114) that is substantially free of undesired fats and lipids. The winterized extract is further processed with a separation (116) process to isolate the cannabinoids (118) and terpenes (120) from one another and the solvent that was used to extract these components. This separation can be accomplished by a variety of means; one method utilizes a rotary evaporator to separate the cannabinoids and terpenes from the solvent, followed by a sequential fractional distillation processes to separate the terpenes and cannabinoids, and further fractional distillation processes (122 and 124) to refine the cannabinoids class and terpenes class of compounds to pure species (126 and 128). The purity of the compound class or individual species is confirmed by chemical analysis (130).

The chemical analysis of the materials may include standard analytical techniques for quantifying the levels of the different chemical components e.g. the cannabinoids and terpenes in the case of cannabis. Other analysis for safety (e.g. ensuring the extracts meet regulations with regards to contaminants such as pesticides, mold and spores, or heavy metals) may be conducted either by the producer or a third party. While it would be most desirable to quantify all of the active compounds present, it may be sufficient to only quantify those components present at levels greater than a certain level (e.g. 0.1%, 0.01%, or 0.001%) by weight in the final product. Examples of analytical techniques for quantifying the potency (i.e. cannabinoids content) and the terpene levels include gas chromatography (GC) or high-pressure liquid chromatography (HPLC). These techniques need to be coupled with appropriate detectors including ultraviolet (UV) absorbance, mass spectrometry (MS), or flame ionizing detector (FID). MS is typically preferred and required for analyzing pesticides and residual solvents as it can also be used to ensure that there are no contaminants are present in the final product. In some cases, it may be necessary to do metals analysis, which can be accomplished using techniques such as inductively coupled plasma atomic emission spectroscopy (ICP-AES). If species fractions do not meet quality specifications, they can be further refined. Cannabinoids (132) and terpenes (134) may be utilized from sources other than the raw cannabis (100). In this case these materials would be refined, and their purity would be confirmed by chemical analysis.

Purified cannabinoids (136), terpenes (138), and other additives (140) that meet a purity specification would then be packaged in cartridges (142) or receiving vials (143) that are individually tagged with an indelible and traceable label that provides an individual serial number and lot number. The label may be comprised of a traditional bar code, a two-dimensional bar code (e.g. a Quick Response or QR code), a three-dimensional barcode (this is similar to the QR code but incorporates colors or a gradient versus just a binary color scheme), a radio-frequency identification (RFID) tag, a nearfield communication (NFC) tag, or other suitable means. Individual cartridge or vial serial numbers, production lots, source information, processing information, third-party safety information, and purity information may be registered in the database of an external control system (144). Cartridges and pre-loaded receiving vials will be designed to be solely compatible for use in an extract formulator (148), which will have a means of reading their individual labels for identification and authentication purposes. The external control system will only allow cartridges and vials to be used in the system if they have been approved, which may include validation of safety and purity by a third party (146).

Additional cartridges filled with additives, flavors, fragrances, solvents, synthetic compounds (including synthetic cannabinoids), diluents, rheology modifiers, preservatives, other natural extracts (such as caffeine, nicotine, alcohols, and other natural products), and other ingredients to tune the properties of the extract or to make other types of extract-based products, as described further below. Cartridges may also be filled with pre-mixed master-batches of any combination of extracts and additives with known composition.

The process shown in FIG. 1 is exemplary; the process for raw cannabis would be similar for other natural products with appropriate modifications such as elimination of unnecessary steps depending on the starting material and the desired natural product.

In one embodiment, the subject natural product is THC. In another embodiment, the subject natural product is CBD. In yet another embodiment, the subject natural products are THC and CBD. In other embodiments the subject natural products within a cartridge are a mixture of compounds of the cannabinoid class that are combined in a manner that is reflective of their typical concentrations in a full spectrum extract of a particular cultivar. In some embodiments the subject natural product packaged in the cartridges are single species of the class of compounds known as terpenes. In other embodiments the are a mixture of compounds of the terpene class that are combined in a manner that is reflective of their typical concentrations in a full spectrum extract of a particular cultivar.

In some embodiments, it may be preferred to package the purified extracts in a “prefilled receiving vial” or “receiving vial” for later use in the extract formulator, as opposed to packaging the purified extract in a cartridge. For example, if the purified extract component tends to crystallize this might make it difficult to dispense at accurate levels in the precise quantities required for a particular formulation without additional features in the extract formulator dispense heads such as carefully controlled heaters and an inert gas blanket to prevent oxidation or the use of expensive dispensing systems for those components. Such complexities can be overcome, and accuracies preserved if instead the material is dispensed from the purification line directly in to receiving vessels with a precise quantity. In these cases, the prefilled receiving vials will only be partially filled with a base of components for a given formulation, e.g. a precise amount of a cannabinoid or mixture of cannabinoids with a precise ratio of components but all of the cannabinoid class of compounds. Packaging cannabinoids in prefilled vials may enable use modalities where the formulator is located in a retail facility and consumers purchase prefilled receiving vials with the cannabinoid they desire and then insert that vial into the formulator to create the desired natural product formulation through the addition of other species and mixing.

Additionally, it may be preferred to package base mixtures of components in the receiving vessel instead of a single pure component, or a mixture of cannabinoids that is reflective of a particular cultivar, for example preloading receiving vials with standard ratios of the cannabinoids THC and CBD with ratios between 1:0 and 0:1 (i.e. THC:CBD). The preloaded receiving vessels would be subject to the same quality standards as the cartridges, registered with the central control system, tracked, and designed to only be compatible with the extract formulator device. Both the cartridges the preloaded receiving vessels may have a means of identification and tracking incorporated, such as RFID tags, bar codes, serial numbers or other means.

These receiving vessels may also be designed such that they are compatible for direct use in a dispensing device, such as a vaporizing device. They may further comprise standardized threads to be compatible with these devices, a reservoir region, integrated heating elements, and means of wicking the natural product formulation from the reservoir region to the heating element or designed in a way such that wicking was not required to bring the extract formulation came in contact with the heating element. These receiving vessels may be provided in a variety of shapes, sizes, and form factors-including, but not limited to those typically available on the market today for use in electronic vaporizing devices. The vessels may also be specifically designed to work only with certain vaporizing devices. Such devices could be further designed to only accept receiving vessels that were filled with the natural products printing system, and these special vessels would only work with the special devices. The device could be comprised of a battery, an LED indicator system, a multifunctional button, a receiving port to connect it with the special vessel, and a means of preventing the use of non-approved cartridges such as an interlock or special threads. The receiving vessels intended for use with these special devices would require a complimentary design feature such as special threads or an interlock key that would only enable them to be compatible with the specially designed devices.

Receiving vessels may be configured to for one-time use products, multiple-use products, or bulk. As such receiving vessels may be configured to collect between 0.1 grams and up to 100 kg.

### Natural-Products Extract Formulator and System

In one aspect, a natural-products extract formulator is provided. In one embodiment, the system includes:
at least two cartridges; wherein each cartridge is configured to contain a purified natural product, a mixture of purified natural products, or additives, and at least one of the natural product extracts is a pure extract that is free of other additives, carriers, solvents, viscosity modifiers, or diluents;
at least two precision dispensers, each configured to dispense the purified natural product from one of the cartridges;
a database of formulations that is located either locally or externally, and which determines how much of each purified natural product is to be dispensed to create each formulation in the database; and
at least one mixer configured to mix the purified natural products dispensed from the at least two precision dispensers; and
a controller that is configured to control dispensation of purified natural products from the natural-products extract formulator by controlling the function of the at least two cartridges, the at least two precision dispensers, the mixer, and at least one temperature within the system.

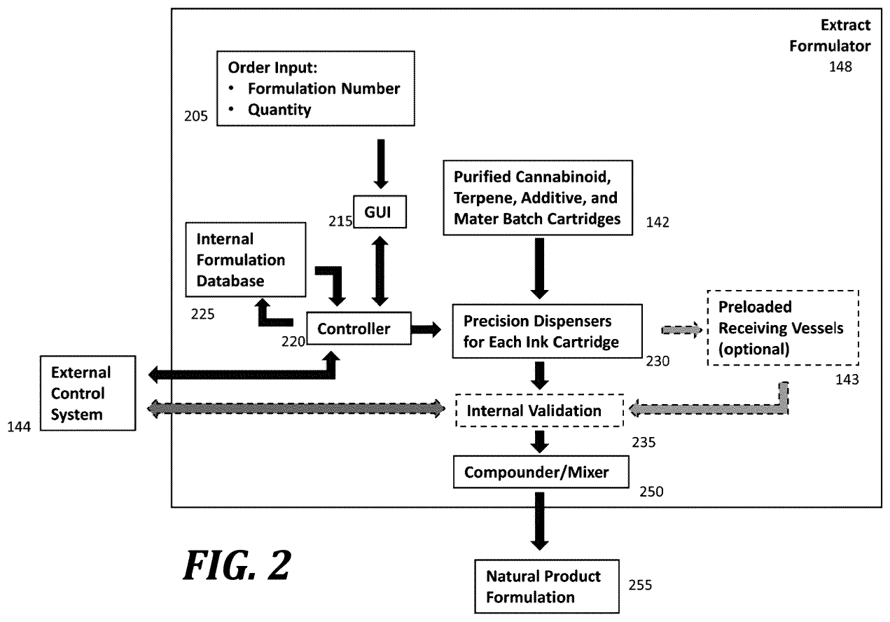

FIG. 2 illustrates an example of an extract formulator (148) that utilizes formulation cartridges (142) to produce uniform and reproducible extract-based products (255). A user inputs commands for an order comprised of formulation number and quantity (205) into a graphical user interface (GUI, 215), for example, which would relay those commands to and internal controller (220). The internal controller references the internal formulation database (225) or optionally a database of formulations on the external control system (148), the controller will report back to the GUI whether the request was approved or if there was an error. If the request is approved the controller will check the system, and if no errors or alerts are found, it will begin to issue commands for the precisions dispensers (230) to dispense the components for the formulation the relevant cartridges into an appropriate receiving vial. Once all of the components have been dispensed the system, will optionally, undergo an internal validation (235) to ensure that the proper components and formulation have been dispensed. If the batch passes the internal validation then the system will mix and all of the components to yield a homogeneous product (255) that is ready for use.

In one embodiment, at least one of the at least two cartridges contains a natural products extract or mixture of natural product extracts from plants in the cannabis genus. In another embodiment, at least one of the at least two cartridges contains a derivative of a natural product. In another embodiment at least one of the at least two cartridges contains a synthetic cannabinoid. The other one or more cartridges may contain a terpene, a mixture of terpenes, or other additives that might be required to make a natural product formulation.

In one embodiment the system and/or natural-products extract formulator further includes a formulation database that includes preset formulations comprising amounts of each of specific natural products to be distributed by the natural-products extract formulator to produce the natural-products formulation, wherein the formulation database is accessible by the controller. The database of formulations contains recipes for different formulations wherein the concentrations for components (e.g. cannabinoids, terpenes, and other additives) are recorded in a normalized fashion (e.g. weight percentage, parts per thousand, parts per million, etc.) that can be readily scaled for any batch size and acceptable tolerances for each component in the final formulation. The formulation may call for the explicit use of specific cartridges (e.g. a specific cartridge that contains a combination of compounds that is reflective of the full spectrum extract for a particular cultivar) or may allow for use of several generic (e.g. cartridges that contain only a single species) cartridges to create formulations. The database will also have a means of cross-referencing which cartridges are preferred for use to create a particular formulation and what acceptable alternatives might be.

In one embodiment, the precision dispensers include a dispensing mechanism that is: the application of positive or negative pressure; physical displacement via a gas or liquid, or change in volume of the reservoir in the cartridge; or pumped from the cartridge via a peristaltic pump, gear pump, displacement pump, or diaphragm pump. In some embodiments of the system the methods of moving and dispensing components include: positive pressure, negative pressure, displacement, pneumatic pumping, hydraulic pumping, peristaltic pumping, piezoelectric pumping, and other methods of inducing fluid flow. In some embodiments the formulation components comprise liquids, solids (including crystalline solids), semi-solids, solutions, gels, resins, slurries, or other forms of matter.

In some embodiments, the temperature of one or more components may be adjusted to facilitate dispensing or mixing; in those embodiments a heating device and a means of controlling the temperature in those regions will be included in the system. In one embodiment, at least one of the at least two cartridges further comprises a heating element and temperature monitoring device. In one embodiment, the temperature-monitoring device is selected from the group consisting of a thermocouple, thermistor, and a resistance temperature detector (RTD). Some purified natural products tend to crystallize, e.g. CBD. In some embodiments it may be preferable to control the temperature of the natural product in the cartridge such that it is maintained above the melting point of the natural product during dispensing, as precise dispensing of a liquid is more readily controlled as compared to the precise dispensing of a crystalline solid. A heating element integrated in to the cartridge would allow for rapid and efficient heating of the natural product. A temperature-monitoring device integrated in to the cartridge near the dispensing port would provide the best means of ensuring that the heating element is achieving and maintaining the target temperatures.

In one embodiment, the heating element integrated into the cartridge is configured to heat material in the cartridge to a temperature of 65° C. or greater. CBD, particularly, melts at about 66° C., so heating to this level is important for pure CBD and CBD-containing compositions. In one embodiment, the heating element integrated into the cartridge is configured to heat material in the cartridge to a temperature in the range of 25-100° C. In one embodiment, the heating element integrated into the cartridge is configured to heat material in the cartridge to a temperature in the range of 25-75° C. In one embodiment, the heating element integrated into the cartridge is configured to heat material in the cartridge to a temperature in the range of 50-75° C. In one embodiment, the heating element integrated into the cartridge is configured to heat material in the cartridge to a temperature in the range of 50-100° C.

In some embodiments of the system, it is preferred that the final formulation be fully or partially mixed. In one embodiment, the mixer is selected from the group consisting of ultrasonication, mechanical mixing, high-shear mixing, vibrational mixing, vortexing, orbital mixing, impinging flow, tortuous path, and centrifugal mixing. Mixing can occur in-line during the dispensing process or after materials have been dispensed into receiving vials. Any and all conventional mixing methods can be used in the disclosed systems. These methods may include, but are not limited to: ultrasonic mixing, vibrational mixing, static mixing, vortexing, microfluidizing, shear mixing, impinging flow, tortuous path, orbital mixing, convective mixing, and other methods known in the art.

In one embodiment, the at least one mixer further comprises a heating element. To ensure proper mixing of the natural products components into a homogenous natural product formulation, the temperature of the mixture may need to be maintained at an elevated temperature sufficient to either melt or dissolve solid components into the rest of the liquid mixture, as discussed above with regard to cartridges. In one embodiment, the heating element integrated into the mixer is configured to heat material in the mixer to a temperature of 65° C. or greater. In one embodiment, the heating element integrated into the mixer is configured to heat material in the mixer to a temperature in the range of 25-100° C. In one embodiment, the heating element integrated into the mixer is configured to heat material in the mixer to a temperature in the range of 25-75° C. In one embodiment, the heating element integrated into the mixer is configured to heat material in the mixer to a temperature in the range of 50-75° C. In one embodiment, the heating element integrated into the mixer is configured to heat material in the mixer to a temperature in the range of 50-100° C.

To ensure that the temperature in the mixer is at the prescribed level a temperature-monitoring device would be employed. In one embodiment, the temperature-monitoring device is selected from the group consisting of a thermocouple, thermistor, and a resistance temperature detector (RTD).

The embodiments above describe the specific device or apparatus that can be used to produce a natural product formulation. In broader terms a system for preparing natural product formulations is described below.

In another aspect, a system for preparing a natural-products formulation is provided. In one embodiment, the system includes:
at least two purified natural product extracts;
a natural-products extract formulator as disclosed herein; and
a receiving container or vial;
wherein the system is configured to dispense the at least two purified natural product extracts in precise quantities and mixed at an appropriate temperature and with sufficient agitation to yield a homogenous natural product formulation.

In one embodiment the system further includes a graphical user interface configured to accept input from a user to control the operation and output of the natural-products extract formulator of any of the previous claims and to monitor the output of natural product formulation produced by the natural-products extract formulator. The system is comprised of a graphic user interface (GUI), an internal controller, an external control system, formulation cartridges, precision dispensing heads for each cartridge, and a means of mixing or compounding the formulation after dispensing each of the components. The system may also optionally have means of controlling the temperature of each formulation cartridge for stability purposes or for controlling the viscosity of the components.

The internal control system or internal controller controls the internal functions of the extract formulator. The external control system or external controller provides the independent oversight that ensures quality, safety, purity, and is key to the reliability, consistency and brand integrity of the products produced with the system. Extractors fill cartridges or receiving vials with natural product extract or purified extracts, register the lot with the external control system. The external control system can track individual cartridges or vials through validation (e.g. by a third-party lab) and ultimate release. Lots can be allowed or prevented from release/use by the external control system based on such validation and confirmation of provenance. The extract formulator and its internal control system will only permit use of receiving vials and cartridges that are approved by the external control system.

Embodiments of the system are contemplated for use in various scenarios, which may each demand different batch sizes to be produced, and therefore a different model of system. In one embodiment, for use by a large-scale producer, a large batch (i.e. 100-10,000 grams) of a natural product formulation may be produced using the system and methods disclosed herein and then further packaged in to smaller containers for retail sale. In other embodiments, a system may be located at a retail facility and used to produce smaller batches (e.g. 1-10 grams or more) of a natural product on demand or to meet daily sales expectations, for example. Distribution centers or larger retail entities may require systems that fall between these two examples in their production capacity. While the core components and functionality of these systems would be the same the specific components would need to be designed and scaled appropriately to achieve acceptable tolerances for accuracy and reproducibility. In one embodiment, the natural-products extract formulator is designed to dispense 0.5 grams to 5.0 grams of natural-product formulation. In one embodiment, the natural-products extract formulator is designed to dispense 1 gram to 10 grams of natural-product formulation. In one embodiment, the natural-products extract formulator is designed to dispense 10 grams to 100 grams of natural-product formulation. In one embodiment, the natural-products extract formulator is designed to dispense 100 grams to 1000 grams of natural-product formulation. In one embodiment, the natural-products extract formulator is designed to dispense 500 grams to 5000 grams of natural-product formulation. In one embodiment, the natural-products extract formulator is designed to dispense 1000 grams to 10,000 grams of natural-product formulation. In one embodiment, the natural-products extract formulator is designed to dispense 10 kilograms to 100 kilograms of natural-product formulation.

In some embodiments, the system may further include a cartridge or reservoir filled with a flushing solution that could be used to purge and flush the system between batches. This provides a means of reducing or eliminating the potential for cross-contamination of a dispensing run with components from those of a previous dispensing run. In some embodiments, the system may further include a means of handling a waste stream generated by flushing, purging, or cleaning the system (e.g. a waste reservoir and the appropriate plumbing).

In one embodiment, the natural-products extract formulator further includes means of recognizing and tracking cartridges and receiving vials via radio-frequency identification (RFID), near-field communication (NFC), or optical methods. The marking of the cartridges and receiving vials with indelible markings allows for them to be tracked throughout their lifespan, prevents counterfeiting, provides a means of regulatory control by the external system for disallowing the use of inappropriate, counterfeit, or expired components in a formulation, and provides the user confidence in the integrity of the system and products (i.e. natural product formulations) that it produces. The system will integrate a means of reading the markings, tags, or labels on the cartridges and vials appropriate for the markings, tags, or labels for which it will be compatible. For bar codes, the reader will be comprised of a standard bar code scanner. For two- and three-dimensional bar codes or quick response (QR) codes the system would integrate image capture and processing capabilities. For RFID and NFC tags the system would integrate the appropriate readers and antenna for the protocols that are being used. In all cases the reading system would be designed to communicate with the system controller, which would be able to further communicate with the external control system for validation and authorization purposes.

In one embodiment, the natural-products extract formulator can be validated to meet Good Manufacturing Practice (GMP) standards for pharmaceutical formulations. In some embodiments the system will be designed such that it can produce pharmaceutical grade formulations, as such the system will be designed to meet Good Manufacturing Practice (GMP) standards. When combined with cartridges that have been fabricated according to GMP standards that are filled with natural products and additives that have also been processed according to GMP standards, the system would be able to reproducibly dispense pharmaceutical grade products that meet GMP standards.

### External Control Systems

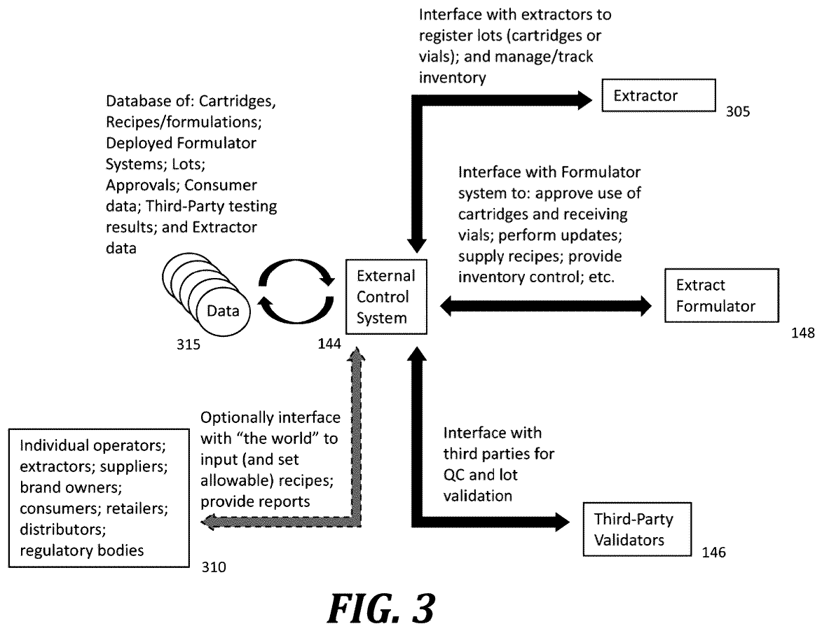

FIG. 3 illustrates how the external control system interacts with the other various components of the ecosystem. In one embodiment, to produce a product a user would input the desired formulation number and the quantity to be produced; the natural products formulator would then dispense the appropriate amounts of each component into a vessel, mix them, and then dispense or release the final product. In another embodiment, an external control system is provided that communicates with a natural-products extract formulator and/or system as disclosed herein and is configured to authorize components to be dispensed and mixed to form predetermined formulations comprising a natural-products formulation.

The external control system (144) will continuously interact with all of the natural product extract formulators (148) that have been deployed. In one embodiment, the external control system includes a component configured to track and/or trace the predetermined formulations, including for third party validation, including by authorities and/or regulators. The external control system will review the requests from the natural product extract formulators and authorize or deny the requests based on a variety of factors including but not limited to authority to use the requested formulation, validation that the appropriate cartridges and vials are present in the formulator to create desired formulation and that they have not expired, validation that the requested formulation meets local regulations, and that there is sufficient quantities of materials in the cartridges to create the requested formulation.

In one embodiment, the external control system further includes a component configured to push updates, perform system health monitoring, lock out users, apply permissions, and/or perform cartridge usage monitoring. In cases where the system finds an issue with a particular request it will have the ability to prevent a requested formulation from being dispensed. Some examples of issues that could lead to the external control system denying a request include: an unauthorized formulation (i.e. the user had not procured access to use that particular formulation), the user does not have the proper legal authority to produce the formulation registered in the external database, the formulation would not conform to local regulations, a cartridge has insufficient material to produce the desired formulation, in the quantity requested, one or more component cartridges has expired, the amount of material in one of the cartridges does not match the usage profile in the database implying that it has been tampered with, one of the cartridges is registered to a different user or location or formulator, the receiving vial is not a detected, the receiving vial is not registered in the data base of the external control system, or the external data base cannot establish a connection to the appropriate reporting authority to notify them of the transaction. These examples illustrate some situations that could lead to the external control system preventing a requested formulation form being dispensed, but is not exhaustive, and many other conditions, errors, or situations could also lead to a dispense denial.

In one embodiment, the external control system further includes an associated database of possible predetermined formulations. If the requested formulation and quantity is authorized for the requestor and all of the components cartridges are present with sufficient supplies for the request, and all component cartridges are not expired the external control system will authorize the request and allow it to proceed. In one embodiment, the external control system further includes a component configured to track dispensed predetermined formulations. This feature allows for automated reporting and tracking to authorities or authorized tracking agencies for regulatory or tax purposes. In one embodiment, the external control system further includes a component configured to track origins of components dispensed. This feature enables the external control system to generate logistics and sales reports in the case of adverse events that may necessitate a product recall to facilitate an expeditious response. In one embodiment, the external control system further includes a component configured to track an amount of THC dispensed in a predetermined formulation. Some jurisdictions may only want to track sales of THC for tax and regulatory purposes, this feature would enable that.

In some embodiments, the natural products formulator, cartridges, and vials will be designed to meet good manufacturing practices (GMP) standards, and the products dispensed from them will also meet GMP compliance standards for pharmaceutical grade formulations as validated by third-party validators (146). In other embodiments the third-party validators (146) only validate the concentrations, purity, safety of the contents of the cartridges and vials, and reproducibility of the natural products formulator. In all cases the external ecosystem maintains a record of the third-party validations for cartridges, vials, and natural products formulator registered in the system and only allows registered cartridges, vials, and natural products formulator to dispense product (255).

The external control system (144) will collect data (315) on the entire ecosystem. This database will contain a record of all cartridges, vials, production lots, and extract formulators systems that are registered in the ecosystem. It will also maintain a record of approved formulations, third-party validations for all ecosystem components. Natural product extractors who extract, purify, refine natural products and package the pure species in cartridges (142) and vials (143) for use in the natural product formulator (148) will interact with the external controls system (144) to register production lots, provide third-party validation information for their production lots, and to manage and track inventory distribution. Third-party validators (146) will interact with the external control system (144) to acknowledge receipt of samples for testing and to provide validation-testing results to the external control system and extract producers.

The external control system may additionally, interface to the rest of the world in several ways. It may provide access to data (315) such as production and consumer data to entities such as individual operators, extractors, suppliers, brand owners, retailers, distributers, and regulatory bodies (310). It will also provide a platform for new formulations to be submitted, registered and purchased.

The natural product extract formulator may further comprise means of verifying what was dispensed to the vials via mass, spectroscopic, chromatographic, or optical methods. The system may include a validation system, such as a load cell or balance that monitors the mass of the receiving vessel and ensures that the appropriate amount of each component was added according to the formulation specifications and would alarm if there was a discrepancy. Such a monitoring system could also be used for inventory control and to assess operational fitness of the equipment. Other methods may be used to validate or monitor dispensing including but not limited to electrical measurements, impedance measurements, pressure measurements, thermal measurements, capacitance measurements, or any such measurements employing commercially available flow control sensors. Additional means of verifying the composition of the formulation that was dispensed would include chemical analysis by standard means such spectroscopic or chromatographic means, wherein results for the dispensed formulation would be compared to those in a database for each formulation.

The quality control of the formulation cartridges will enable the production of extract-based products with pharmaceutical grade control of the reproducibility. The purity and composition of every lot of cartridges to be used in the extract formulator can be validated by a third party, labeled and tracked by the producer, and authorized by the external authorization controller prior to sale or use. Individual cartridges and receivers will be labeled with indelible identification tags, such as RFID tags, and registered with the central authorization control center. The use of cartridges will be tracked and authorized by the central authorization via the external control system and optionally reported out to appropriate authorities for tracking, security, and tax purposes. For this purpose, the system will be equipped a means of digital communication with the external control system—for example, using wireless or wired connection to the internet or cellular capabilities.

This digital communication will allow direct monitoring, intervention, and control of the system off-site. The external control system will maintain a database of possible formulations, which will allow new natural product formulations, vessels, or cartridges to be uploaded to the system. Additionally, this control system will be able to push updates or lockout functions. The control center can be used to validate “approved” cartridges for use. The control center can also enable the ability to alert and/or automatically re-order consumables and cartridges as needed. The external control system can also disable cartridges or vessels from use. This may be desirable, for example, after their expiration date, or if their mass/volume changes significantly relative to how the cartridge has been used in the extract formulator i.e. a means of detecting when formulation cartridges have been tampered with and deactivating them from use in those cases. FIG. 3 illustrates how the external control system operates in the ecosystem.

The internal controller can be configured to send or receive data from the external control system or remote server. The data can include, for example, equipment status, reports on each batch, which cartridges have been used and the amount of material dispensed from each cartridge, requests for additional deliveries, or new or updated recipes or chemical formulation data. In an embodiment, a retailer, distributor, consumer, or brand owner can send recipes via the Internet, or other network connection, to the system such that the recipes are electronically loaded into an embodiment of the disclosed extract formulator at a desired retail or distribution location. Additionally, an extractor or supply company can communicate with an embodiment of the extract formulator to update, recall, or change any one of the plurality of different recipes in the system that utilize component formulations supplied by the extractor or supply company.

An embodiment of the present invention comprises one or multiple databases as containing information such as: formulation compositions, formulation methods, security passwords, user information, preferences, location data, time data, or other digital information. Such databases may be maintained local to the formulation system or remotely (e.g. associated with an external control system or in the cloud).

An embodiment of the present invention includes a Graphical User Interface (GUI) that can provide real-time reporting, inventory status and information, cartridge status reporting and alert notifications, along with the interface to configure recipes and batches. The GUI provides a local operator with the capability to configure the system for operation and to monitor the process. The GUI can also provide an interface to initiate or review updates to recipes that are downloaded from a remote location via the system's network connection. The GUI can also provide an interface to enter or configure a customized recipe at the local installation of the system.

An embodiment of the present invention includes additional security and safety features to prevent unauthorized use. For example child- and tamper-proof enclosures can be used to prevent operation or dispensing. Authorized users could be identified by, for example, RFID/NFC technology, a password, physical key, key card, biometric, or other security feature. Multiple levels of security can be used to enable multiple classes of users to restrict access to certain system features such as cartridge installation, maintenance, formulation programming, or general use. In preferred embodiments the cartridge enclosure for supply cartridges would be lockable to prevent general access after cartridges were installed.

An embodiment of the present invention includes a method of preparing in-depth detailed reports on all processes from customer input, applied formulations, additives, inventory-level control of products, selected recipes, and automatic replenishment of consumable components from appropriate distribution centers. The data sharing capability of an embodiment of the invention provides for communication between individual operators of systems, extractors and suppliers, brand owners, consumers, and retailers and distributors.

In an embodiment, a retailer, distributor, consumer, or brand-owner can specify recipes via the Internet, or other network connection, to the system such that the recipes are electronically loaded into an embodiment of the disclosed extract formulator at a desired retail or distribution location. Additionally, an extractor or supply company can communicate with such an extract formulator to update, recall, or change any one of the plurality of different recipes in the system that utilized component formulations supplied by the extractor or supply company.

Specialized equipment that is designed to fill, mark or label, and package the formulation cartridges or prefilled receiving vessels are also contemplated. This equipment may for example, fill the cartridge or vessel with a prescribed amount of material, cap or seal the vessel or cartridge, and then apply an indelible identifier, such as an RFID tag, and log it in a database with the rest of the vessels or cartridges from that lot. It is contemplated that such a database would simplify tracking, quality control, safety, surety, validation (e.g. by a third party), and ultimate release for sale.

A means of cataloging compositions is further contemplated, whereby the specific composition of a formulation would be represented by a number, the Formulation Number. The formulation number would actually be a string of numbers, letters, and potentially other characters such as periods, commas, slashes, or dashes used to describe the exact composition of a formulation.

### Natural-Product Formulation Cartridge

In another aspect, a cartridge is provided that is configured to be used in a natural-product extract formulator as disclosed herein. In one embodiment, the cartridge can be certified to meet Good Manufacturing Practice (GMP). In an embodiment of the present invention cartridges for use with the system may be equipped with additional integrated functionalities. Such functionalities may include, but are not limited to: RFID or NFC recognition tags, heaters, dispensers, integrated circuits, a locking function, and/or a fail-safe to prevent tampering or unapproved refilling.

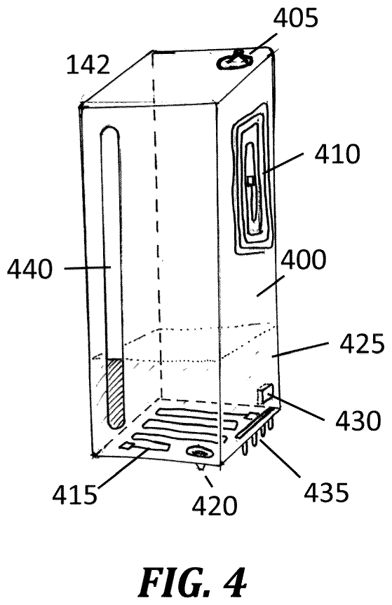

FIG. 4 illustrates one example of a cartridge for the natural product formulator. The cartridge (142) is comprised of a container with an internal reservoir (400), a tamper-proof inlet (405), an RFID identifier (410), a resistive heater with integrated thermocouple (415), a tamper-proof outlet (420), the purified natural product, masterbatch mixture, additive, or flushing solution (425), sensor (430), electrical contactor pins (435), and an observation window (440). The tamper-proof inlet allows for the extract producer to fill the cartridges with purified natural product, masterbatch mixture, additive, or flushing solution but prevents further addition or removal of materials after being sealed. In one embodiment, the natural product extract is an extract from a plant in the cannabis genus.

In one embodiment, the cartridge further includes means of recognizing and tracking cartridges and receiving vials via radio-frequency identification (RFID), near-field communication (NFC), or optical methods. As should be appreciated by those skilled in the art, it should be noted that NFC is a subset of RFID technology. The RFID identifier or tag allows for the cartridge to be interrogated in a wireless manner without the requirement of direct line of sight, physical, or electrical contact, and further allows for simultaneous inventorying of many tags. The RFID tags also advanced security features, such as unique identification numbers and many encoded memory sectors that can make counterfeiting very difficult, if not impossible.

In one embodiment, the cartridge further includes a heating element and temperature-monitoring device such as a thermocouple, thermistor, or resistance temperature detector (RTD). A resistive heater and an integrated thermocouple allow for the temperature of the natural product or additive in the internal reservoir to be maintained at a level that ensures accurate dispensing. For some materials this means a temperature that induces a change in state of the natural product, i.e. melting of a solid to a liquid. In other cases the prescribed temperature does not change the state of the materials in the reservoir (i.e. it remains a liquid), but reduces the viscosity of the substance such that it can be readily and accurately dispensed and mixed by the system. The tamper-proof outlet allows materials to be dispensed by the dispensing mechanism but not by other means and does not allow for the addition of materials. The cartridge may also incorporate sensors. A sensor may monitor the temperature of the cartridge, or monitor the properties of the contents due to contamination or degradation. The sensor may also just interact with the formulator to indicate information about the cartridge and its contents.

The electrical contactor pins allow for an electrical connection to be made between the cartridge and the formulator for various functions, e.g. providing power to the heater, collecting signals form the temperature monitoring device, and reading the RFID chip in contact mode.

The observation window allows for the user to examine the contents of a cartridge to gauge how much materials is left in the cartridge, and if there are any noticeable changes in appearance, e.g. changes in color, separation, or precipitation.

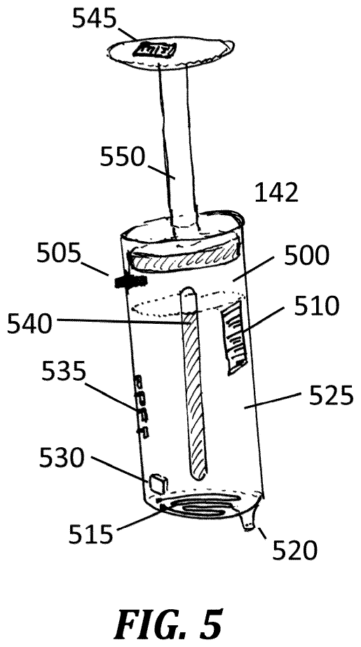

In one embodiment, the cartridge is a syringe or a cylinder with an integrated chaser. FIG. 5 shows and illustration of such an embodiment, which is functionally similar to the cartridge in FIG. 4 except for the dispensing mode being primarily limited to physical displacement via the plunger or chaser. The cartridge (142) is comprised of a container with an internal reservoir (500), a tamper-proof lock pin (505), an RFID identifier (510), a resistive heater with integrated thermocouple (515), a tamper-proof outlet (520), the purified natural product, masterbatch mixture, additive, or flushing solution (525), sensor (530), electrical contactor pins (535), an observation window (540), an product label (545), and an integrated displacement dispensing plunger (550).

One difference between the cartridge in FIG. 5 compared to that in FIG. 4, is that the cartridge in FIG. 5 incorporates a tamper-proof lock pin that prevents unauthorized dispensing, or unauthorized materials from being added or removed from the cartridge.

### Methods of Manufacturing Natural-Product Formulations

In another aspect, a method of manufacturing a natural-products formulation is provided and includes forming a natural-products formulation using a natural-products extract formulator and/or system as disclosed herein. The process for preparing a purified extract and packaging it in a cartridge or prefilled receiving vial is described above and in FIG. 1. The process for using cartridges, a formulator, and a receiving vial and the interactions throughout that process with an external control system are described above and in FIG. 2 and FIG. 3. Generally the method comprises providing a raw material and processing it to a purified extract, providing a cartridge or receiving vial and packaging the purified extract in a cartridge or pre-filled receiving vial as described above; providing cartridges, a natural product formulator, and receiving vials as described above, and using the natural product formulator as described above to dispense a natural product formulation.

In one embodiment, the method includes a step of heating a purified extract or mixture to an elevated temperature to ensure proper mixing of the natural products components into a homogenous natural product formulation. The temperature of the mixture may need to be maintained at an elevated temperature sufficient to either melt or dissolve solid components into the rest of the liquid mixture, as discussed previously. In one embodiment, the heating is to a temperature of 65° C. or greater. In one embodiment, the heating is to a temperature in the range of 25-100° C. In one embodiment, the heating is to a temperature in the range of 25-75° C. In one embodiment, the heating is to a temperature in the range of 50-75° C. In one embodiment, the heating is to a temperature in the range of 50-100° C.

In one embodiment the heating is via a heating element in one or more of the cartridges. In another embodiment, the heating is via a heating element in the mixer. In yet another embodiment, the heating is via heating elements in both the mixer and at least one cartridge.

### Natural Product Formulations

In another aspect, a natural-products formulation is provided that is formed using the methods disclosed herein. The natural product formulation is comprised of martials dispensed from the at least two cartridges in the formulator as described above, wherein at least one of the materials is a purified natural product extract. The chemical profile of a natural product formulation could be based on the average profile obtained upon analysis of a particular cultivar or finished product from a particular cultivar, or it could be defined for a custom or “ideal” formulation. The final product may include other ingredients to make up the final product. For example, a topical formulation may include emollients, solvents (including water), surfactants, perfumes, thickeners, emulsifying agents, and natural oils and butters beyond those extracted from the raw plant materials. Formulations meant for vaporizing may include other ingredients such as propylene glycol, glycerin, or water. Additional additives include propylene glycol, vegetable glycerin, wetting agents, flavors, diluents, rheology modifiers, other natural extracts (e.g., nicotine, caffeine, alcohol), preservatives, combinations thereof, and the like. Formulations meant for edible consumption may include other ingredients that are typically found in, but not limited to, beverages, candies, butters, mints, gums, chewables, tinctures, cookies, and other edible products.

While illustrative embodiments have been illustrated and described, it will be appreciated that various changes can be made therein without departing from the spirit and scope of the invention.

The embodiments of the invention in which an exclusive property or privilege is claimed are defined as follows:

1. An external control system that communicates with a natural-products extract formulator and/or system configured to authorize components to be dispensed and mixed to form predetermined formulations comprising a natural-products formulation.

2. The external control system of claim 1, further comprising a component configured to track and/or trace the predetermined formulations, including for third party validation, including by authorities and/or regulators.

3. The external control system of claim 1, further comprising a component configured to push updates, perform system health monitoring, lock out users, apply permissions, and/or perform cartridge usage monitoring.

4. The external control system of claim 1, further comprising an associated database of possible predetermined formulations.

5. The external control system of claim 1, further comprising a component configured to track dispensed predetermined formulations.

6. The external control system of claim 1, further comprising a component configured to track origins of components dispensed.

7. The external control system of claim 1, further comprising a component configured to track an amount of THC dispensed in a predetermined formulation.
